(19) United States
(12) Patent Application Publication
(10) Pub. No.: US 2025/0234499 A1
(43) Pub. Date: Jul. 17, 2025

(54) ELECTROSTATIC DISCHARGE PROTECTION FOR WEARABLE DEVICES

(71) Applicant: Oura Health Oy, Oulu (FI)

(72) Inventors: Heikki Juhani Huttunen, Haukipudas (FI); Reetta Sofia Kuonanoja, Oulu (FI); Marko Uusitalo, Oulu (FI); Kari Kuisma Kanniainen, Ii (FI)

(21) Appl. No.: 19/016,793

(22) Filed: Jan. 10, 2025

Related U.S. Application Data
(60) Provisional application No. 63/619,865, filed on Jan. 11, 2024.

(51) Int. Cl. H05K 9/00; H01Q 1/27; H05K 5/00; H05K 5/02

(52) U.S. Cl. CPC H05K 9/0067; H01Q 1/273; H05K 5/0086; H05K 5/0247

(57) ABSTRACT

Methods, systems, and devices for protecting wearable devices from electrostatic discharge are described. A wearable device may include an inner housing and an outer housing that include electrically-conductive materials. The wearable device may include a printed circuit board (PCB) disposed at least partially between the inner housing and the outer housing, the PCB including one or more sensors configured to acquire physiological data from the user through one or more apertures within the inner housing. The wearable device may include an electrical connection component configured to reduce a difference in an electrical potential between the inner housing and the outer housing by electrically coupling the inner housing and the outer housing.

### CROSS REFERENCE

The present application for patent claims the benefit of U.S. Provisional Patent Application No. 63/619,865 by HUTTUNEN, entitled “ELECTROSTATIC DISCHARGE PROTECTION FOR WEARABLE DEVICES,” filed Jan. 11, 2024, assigned to the assignee hereof, and expressly incorporated by reference herein.

### FIELD OF TECHNOLOGY

The following relates to wearable devices and data processing, including techniques for electrostatic discharge protection for wearable devices.

### BACKGROUND

Some wearable devices may be configured to collect data from users, including temperature data, heart rate data, and the like. In some cases, the wearable devices may include metallic materials and may be subject to electrostatic discharge, which may “shock” the user, damage components of the wearable device, or both.

### BRIEF DESCRIPTION OF THE DRAWINGS

FIG. 1 illustrates an example of a system that supports techniques for electrostatic discharge protection for wearable devices in accordance with aspects of the present disclosure.

FIG. 2 illustrates an example of a system that supports techniques for electrostatic discharge protection for wearable devices in accordance with aspects of the present disclosure.

FIG. 3A and FIG. 3B show examples of diagrams that support techniques for electrostatic discharge protection for wearable devices in accordance with aspects of the present disclosure.

FIG. 4 shows an example of a diagram that supports techniques for electrostatic discharge protection for wearable devices in accordance with aspects of the present disclosure.

FIG. 5 shows an example of a diagram that supports techniques for electrostatic discharge protection for wearable devices in accordance with aspects of the present disclosure.

### DETAILED DESCRIPTION

Some wearable devices may be manufactured with metal or other electrically-conductive components. For example, a wearable device (e.g., a wearable ring device) may include an inner metal housing (e.g., inner cover, inner shell) and an outer metal housing (e.g., outer cover, outer shell) that enclose components of the wearable device. In some examples, the inner metal housing, the outer metal housing, and internal components (e.g., a printed circuit board (PCB), antenna component, etc.), may be electrically isolated from each other using one or more insulating materials. For instance, the inner and outer metal housings may be separated and isolated from one another using insulating components in order to allow wireless signals to propagate into or out of the wearable device. However, the electrical (e.g., galvanic) isolation of the respective conductive components may result in the conductive components developing different electrostatic charges or potentials. For example, the inner metal housing, which may be in contact with a user, and the outer metal housing, which may be exposed to the user's surroundings, may develop different electrostatic potentials. These varying electrostatic potentials may result in electrostatic discharge (ESD) and dielectric breakdown between the inner and outer housings, thereby resulting in a strong electric current flowing through the wearable device, which may damage internal components of the wearable device and/or “shock” the user.

In accordance with examples as described herein, a wearable device may include an electrical connection component (e.g., a galvanic component, a metal component) to prevent dielectric breakdown and damage to the internal components of a wearable device due to ESD. In some examples, the electrical connection component may electrically (e.g., galvanically) couple an inner metal housing and an outer metal housing of a wearable device. As such, the electrical connection component may enable electrical current to flow between the inner and outer housings, thereby maintaining the inner metal housing and the outer metal housing at the same electrostatic potential and preventing dielectric breakdown (e.g., causing ESD).

In some examples, the electrical connection component may be positioned within the wearable device to avoid damage and/or interference with the internal components of the device, such as antenna components. For example, the electrical connection component may be positioned some distance away from an antenna component, where the distance may be based on a wavelength associated with the antenna component (e.g., a quarter wavelength away), which may reduce interference with the antenna component. In some examples, the electrical connection may be or include conductive metal clips, metallic “pins” or screws that contact the inner and outer housings, welding (e.g., soldering) components between the inner and outer housings, a conductive foam material, or a combination thereof.

Aspects of the disclosure are initially described in the context of systems supporting physiological data collection from users via wearable devices. Aspects of the disclosure are additionally illustrated in the form of diagrams illustrating wearable devices that support ESD protection. Aspects of the disclosure are further illustrated by and described with reference to apparatus diagrams, system diagrams, and flowcharts that relate to electrostatic discharge protection for wearable devices.

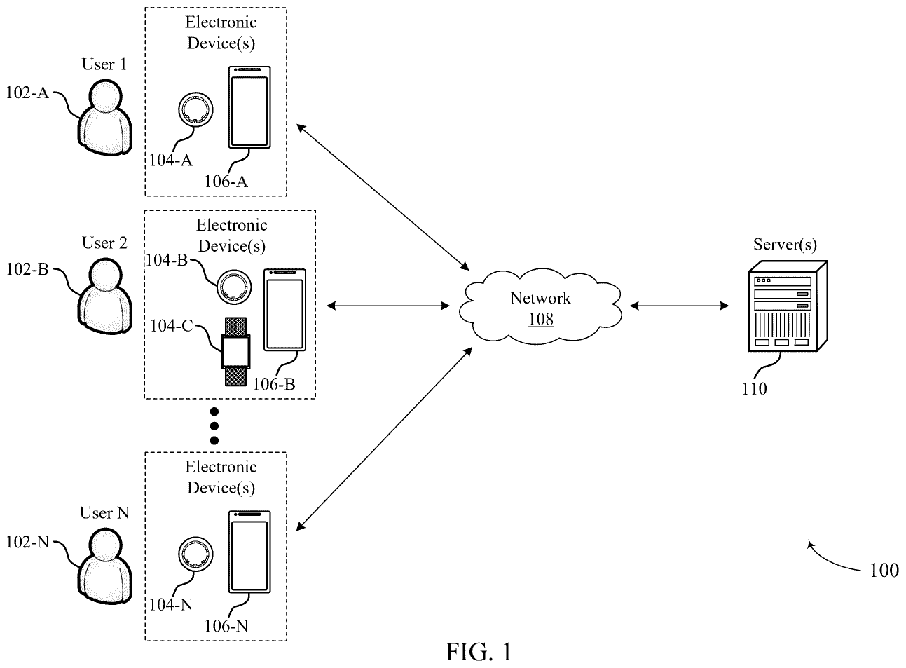

FIG. 1 illustrates an example of a system 100 that supports techniques for electrostatic discharge protection for wearable devices in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for preventing dielectric breakdown and damage to the internal components of a wearable device 104 due to ESD. For example, a wearable device 104 may include an electrical connection component (e.g., galvanic component) that may electrically (e.g., galvanically) couple an inner metal housing and an outer metal housing of a wearable device 104. In some examples, the electrical connection component may be or include conductive metal clips, metallic pins or screws that contact the inner and outer housings, welding (e.g., soldering) components between the inner and outer housings, a conductive foam or other conductive material/substance, or a combination thereof. For example, the electrical connection component may include a metallic screw and a metallic spring component configured to electrically couple the inner housing and the outer housing. As such, the electrical connection component may enable electrical current to flow between the inner and outer housings, thereby maintaining the inner metal housing and the outer metal housing at a same electrostatic potential and preventing dielectric breakdown (e.g., that may cause ESD damage). In some examples, a positioning of the electrical connection component may be based on components of the wearable device 104. For example, the electrical connection component may be positioned away from an antenna element of the wearable device 104 based on a wavelength associated with an antenna element.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

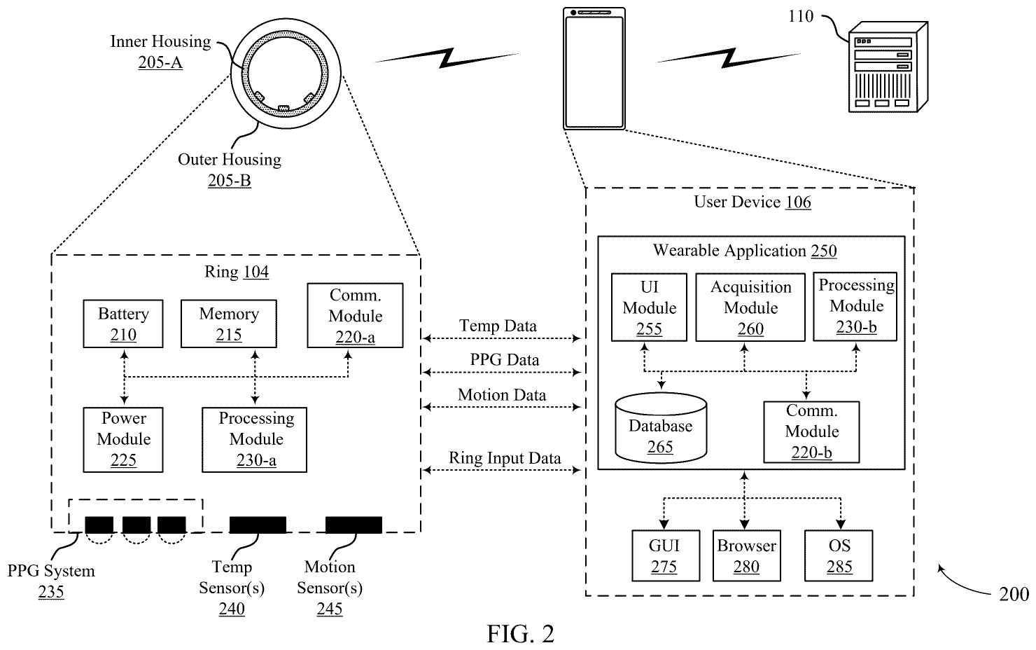

FIG. 2 illustrates an example of a system 200 that supports techniques for electrostatic discharge protection for wearable devices in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more PCBs, such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some cases, the inner housing 205-a (e.g., inner ring-shaped housing, inner cover, inner shell) and the outer housing 205-b (e.g., outer ring-shaped housing, outer cover, outer shell) may include metal or other electrically-conductive materials or components. In some examples, the inner housing 205-a, the outer housing 205-b, and internal components, such as a PCB or an antenna component, may be electrically isolated from each other using one or more insulating materials. For instance, the inner housing 205-a and the outer housing 205-b may be separated and isolated from one another using insulating components in order to allow wireless signals to propagate into (and out of) the ring 104. However, the electrical isolation of the respective conductive components may result in the conductive components developing different electrostatic charges or potentials. For example, the inner housing 205-a, which may be in contact with a user, and the outer housing 205-b, which may be exposed to the user's surroundings, may develop different electrostatic potentials. These varying electrostatic potentials may result in ESD and dielectric breakdown between the inner housing 205-a and outer housing 205-b, thereby resulting in a strong current flowing through the ring 104, which may damage internal components of the ring 104.

In accordance with examples as described herein, a ring 104 may include an electrical connection component (e.g., a galvanic component, a metal component) to prevent dielectric breakdown and damage to the internal components of a ring 104 due to ESD. In some examples, the electrical connection component may electrically (e.g., galvanically) couple the inner housing 205-a and the outer housing 205-b of a ring 104. As such, the electrical connection component may enable electrical current to flow between the inner and outer covers, thereby maintaining the inner housing 205-a and the outer housing 205-b at a same electrostatic potential and preventing dielectric breakdown (e.g., that may cause ESD damage). In some examples, the electrical connection component may be positioned within the ring 104 to avoid damage or interference with the internal components of the device, such as antenna components. For example, the electrical connection component may be positioned some distance away from an antenna component, where the distance may be based on a wavelength associated with the antenna component (e.g., a quarter wavelength away), which may reduce interference with the antenna component. In some examples, the electrical connection may be or include conductive metal clips, metallic “pins” or screws that contact the inner and outer covers, welding (e.g., soldering) components between the inner and outer covers, or a combination thereof.

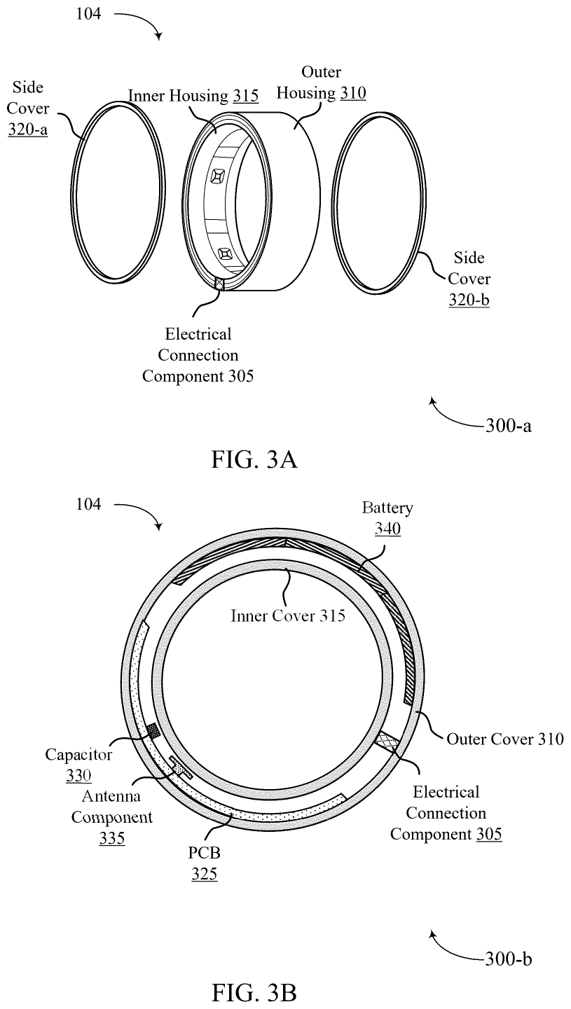

FIG. 3A and FIG. 3B show examples of a diagram 300-a and a diagram 300-b that supports techniques for electrostatic discharge protection for wearable devices in accordance with aspects of the present disclosure. The diagram 300-a and the diagram 300-b illustrate examples of a wearable ring device 104 (e.g., a ring 104) that include an electrical connection component 305 (e.g., an galvanic component), in accordance with examples as described herein.

In some cases, an inner housing 315 (e.g., an inner cover, inner shell, inner housing component) and an outer housing 310 (e.g., an outer cover, outer shell, outer housing component) of the wearable ring device 104 may be manufactured from electrically-conductive materials, such as metallic materials. For example, the inner housing 315 and the outer housing 310 may be or include a same metallic material or a different metallic materials. As shown in FIGS. 3A and 3B, in the context of a finger-worn wearable ring device 104, the inner housing 315 and the outer housing 310 may approximate the shape of a ring (e.g., ring-shaped housings) that are configured to at least partially wrap around/surround a finger of the user. In some cases, the wearable ring device 104 may further include side covers 320-a, 320-b. In some cases, the side cover 320-a and a side cover 320-b may be manufactured from non-electrically conductive materials (e.g., plastic materials, non-metal materials), which may allow for wireless signals (e.g., high-frequency wireless signals) to propagate into and out of the wearable device to support communications with other devices. Additionally, insulating materials (e.g., epoxy materials) may be used within the wearable ring device 104, thereby electrically insulating the inner housing 315 from the outer housing 310.

In some cases, the inner housing 315 and the outer housing 310 may develop different electrical charges (e.g., potentials) due to the inner housing 315 and the outer housing 310 being electrically insulated from each other. As the outer housing 310 and the inner housing 315 are positioned relatively closely in the wearable ring device 104, a difference in potential (e.g., in accumulated charge) between the inner housing 315 and the outer housing 310 may cause dielectric breakdown, where insulating components between the inner housing 315 and the outer housing 310 may temporarily act as conductors. While dielectric breakdown may not occur for large devices where the inner housing 315 and the outer housing 310 are relatively far from each other, the wearable ring device 104 may exhibit relatively small distances between the inner housing 315 and the outer housing 310 (e.g., to support a slim form factor of the wearable ring device 104), and dielectric breakdown may therefore be problematic.

For example, the outer housing 310 may be exposed to a surrounding environment of a user, while the inner housing 315 may (e.g., at least partially) contact a tissue (e.g., skin) of the user. The exposure of the outer housing 310 and the inner housing 315 to the surrounding environment and the tissue of the user, respectively, may result in the outer housing 310 and the inner housing 315 developing different electrostatic potentials, which may result in ESD and dielectric breakdown. When dielectric breakdown occurs, discharge may travel through the insulating materials and any components within the wearable ring device 104, such as a PCB 325, a capacitor 330, an antenna component 335, a battery 340 or other components not shown, which may damage these components. Additionally, or alternatively, an ESD may shock the user, which may be unpleasant for the user. Accordingly, techniques for preventing a large difference in potential between the inner housing 315 and the outer housing 310 may be desired.

In accordance with examples as described herein, the electrical connection component 305 (e.g., galvanic component) may electrically (e.g., galvanically) couple the outer housing 310 and the inner housing 315. Accordingly, an electric potential difference between the inner housing 315 and the outer housing 310 may be reduced (e.g., an electrical potential may be equalized), thereby reducing a likelihood of dielectric breakdown occurring. For example, while a difference in electric potential between the inner housing 315 and the outer housing 310 may occur, current may flow between the inner housing 315 and the outer housing 310 via the electrical connection component 305, thereby protecting other components of the wearable ring device 104 from damage. That is, the electrical connection component 305 may be configured to reduce a difference in electrical potentials (e.g., equalize electrical potentials) between the inner and outer covers, thereby reducing (or preventing) dielectric breakdown. Additionally, ESD that may shock the user may be prevented or reduced in intensity.

In some examples, the electrical connection component 305 may be a pin (e.g., a pogo pin), which may be composed of electrically conductive materials such as brass, copper, or other metal materials. In some examples, the electrical connection component 305 may include a spring assembly (e.g., within the pin, around the pin) that is movable relative to a conductive body of the electrical connection component 305 (e.g., the pin, a screw), which may ensure electrical contact between the inner housing 315, the outer housing 310, and the electrical connection component 305. For example, the conductive body may be coupled (e.g., electrically in contact) with one of the inner housing 315 or the outer housing 310, and the spring assembly may be coupled with the conductive body and the other of the inner housing 315 and the outer housing 310. In some examples, the spring assembly may be in tension, such as to ensure a contact between these components.

The electrical connection component 305 may include different components and may be implemented in a variety of ways. For example, as shown in FIG. 3A, the electrical connection component 305 may be built into one or more side covers 320-a, 320-b of the wearable ring device 104. In such cases, the electrical connection component 305 may include conductive features or traces that are built into the side covers, such as through a molding process, laser direct structuring, etc. In other cases, as shown in FIG. 3B, the electrical connection component 305 may be implemented within a cavity of the wearable ring device 104 between the inner housing 315 and the outer housing 310. In such cases, the electrical connection component 305 may include conductive pins, conductive flanges, conductive springs, screws, conductive foam, other conductive materials, and the like. For example, in some cases, the inner housing 315 and the outer housing 310 may be coupled to one another using a conductive foam material, where the conductive foam material serves as the electrical connection component 305 to reduce/minimize a difference in electrical potential between the inner housing 315 and the outer housing 310. Each of these respective implementations for an electrical connection component 305 are described in further detail herein.

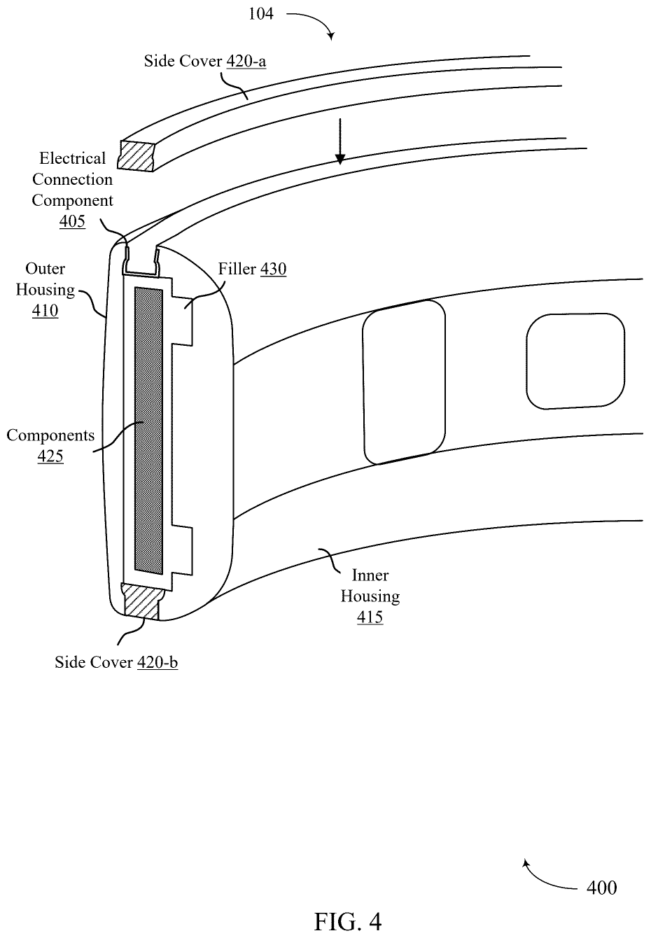

For example, referring to FIG. 3A, the electrical connection component 305 may be disposed within or adjacent to one the side cover 320-a or the side cover 320-b. For example, the side cover 320-a and the side cover 320-b may be or primarily include electrically-insulating materials, where the electrical connection component 305 may be disposed within one of the side cover 320-a or the side cover 320-b, thereby electrically coupling the outer housing 310 and the inner housing 315. In some cases, the electrical connection component 305 may be built into the side cover(s) 320 via laser direct structuring, soldering, welding, using a conductive rubber (e.g., elastomeric connectors such as ZEBRA connectors), or any combination thereof. An example of positioning the electrical connection component 305 within or adjacent to the side cover 320-a or the side cover 320-b is illustrated in more detail with reference to FIG. 4.

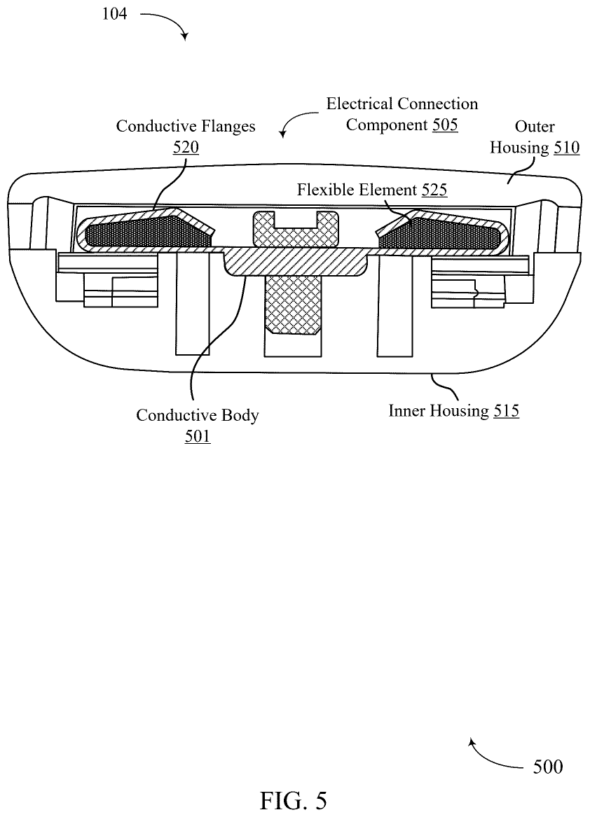

Referring now to FIG. 3B, in additional or alternative cases, the electrical connection component 305 may include one or more conductive bodies that contact (e.g., electrically couple with) one of the inner housing 315 or the outer housing 310. The electrical connection component 305 may also include one or more conductive flanges that contact (e.g., electrically couple with) with the one or more conductive bodies and the other of the inner housing 315 and the outer housing 310. Together, the conductive bodies and conductive flanges may electrically couple the inner housing 315 and the outer housing 310, thereby reducing a difference in electrical potential between the respective covers. In some cases, the conductive flanges may be or include flexible components, such as to exert a mechanical force against the other of the inner housing 315 and the outer housing 310 and against the one or more conductive bodies. An example of the electrical connection component 305 including conductive flanges is described in more detail with reference to FIG. 5.

Additionally, or alternatively, the electrical connection component 305 (as shown in FIG. 3B) may be or include conductive tape, a metal clip, a conductive wrap (e.g., conductive film, conductive foam), a conductive path that may be built on plastic (e.g., of a side over 320 or a filling material of the wearable ring device) using, for example, laser direct structuring, soldering, welding, conductive rubber (e.g., elastomeric connectors such as ZEBRA connectors), a fixed screw, a metal clip, or a combination thereof configured to electrically couple the inner housing 315 and the outer housing 310.

In some examples, a position of the electrical connection component 305 may be selected based on a position of one or more antenna components 335 of the wearable ring device 104, as shown in FIG. 3B. In some cases, for example, an antenna feed of a slot antenna of the wearable ring device 104 may experience interference from the electrical connection component 305. As such, the position of the electrical connection component 305 may be selected to reduce interference to the antenna feed. For example, the electrical connection component 305 may be placed a certain distance from the antenna component 335. In this example, the distance between the electrical connection component 305 and the antenna component 335 may be based on a wavelength or frequency of wireless signals (e.g., a frequency of the wireless signals) communicated via with the antenna component 335.

For instance, the antenna component 335 (e.g., antenna feed of a slot antenna) may be configured to communicate using wireless signals associated with a wavelength of λ. In this example, the electrical connection component 305 may be placed at (e.g., or approximately near) a distance of a quarter of the wavelength (e.g., ¼λ) away from the antenna component 335, or a multiple of half a wavelength thereof

$$( e . g . , \frac{1}{4} λ \pm n \frac{1}{2} λ$$

for an integer nt, such as ¾λ). It has been found that such a positioning of the electrical connection component 305 relative to the antenna component 335 may help reduce interference to wireless communications performed by the antenna component 335.

Continuing with reference to FIG. 3B, in some cases, the electrical connection component 305 may be placed between the PCB 325 and the battery 340. In other words, the electrical connection component 305 may facilitate electrical current to move between the inner housing 315 and the outer housing 310 in a position that is separate or removed from the PCB 325 and/or the battery 340 (e.g., in order to prevent damage to the PCB 325 and/or battery 340). For example, the PCB 325 may span a first radial portion around a circumference of the wearable ring device 104, the battery 340 may span a second radial portion around the circumference, and the galvanic component may be located within a third radial portion between the first radial portion and the second radial portion. Additionally, or alternatively, the electrical connection component 305 may be positioned such as to prevent current from flowing through the PCB 325 or components of the PCB 325. For example, the PCB 325 may span a first portion of a footprint of the wearable ring device 104, and the electrical connection component 305 may be positioned within a second portion separate from the first portion. As such, current that may flow through the electrical connection component 305 may be prevented from flowing through the PCB 325 or components thereof. In some examples, as the positioning of the components may vary depending on a size (e.g., a ring size) of the wearable ring device 104, the positioning of the electrical connection component 305 may be based on the size of the wearable ring device 104.

In some cases, to allow for wireless signals to be transmitted or received by the antenna component 335, the inner housing 315, the outer housing 310, the side cover 320-a, the side cover 320-b, or any combination thereof, may include one or more apertures that enable the wireless signals to pass through the housing of the wearable ring device. Such apertures may enable wireless signals to be communicated to/from the antenna component 335 when the inner housing 315, the outer housing 310, and/or the side covers 320 include electrically-conductive (e.g., metallic) materials. For instance, the inner housing 315, the outer housing 310, the side covers 320, or any combination thereof, may include one or more apertures that are radially aligned with the antenna component 335 (e.g., “windows” that allow for wireless signals to propagate in/out of the wearable ring device 104). The apertures may be filled or covered with non-conductive materials (e.g., plastic) that may allow the wireless signals to be transmitted or received by the antenna component 335. In some cases, the apertures may exhibit a size that is at least half of the wavelength (e.g., −1) associated with the antenna component 335.

While the diagram 300-a and the diagram 300-b illustrate the use of the electrical connection component 305 with respect to a wearable ring device 104, the electrical connection component 305 may be used within other wearable devices 104 that may include electrically-conductive housings. For example, a wrist-worn wearable device may include the electrical connection component 305, as described herein, to electrically couple portions (e.g., an outer housing and an inner housing) of the wrist-worn wearable device.

Accordingly, by including the electrical connection component 305 within the wearable ring device 104, the outer housing 310 and the inner housing 315 may be electrically coupled, thereby reducing the risk of dielectric breakdown and ESD.

FIG. 4 shows an example of a diagram 400 that supports techniques for electrostatic discharge protection for wearable devices in accordance with aspects of the present disclosure. The diagram 400 illustrates an example cross-section of a wearable ring device 104 that incorporates an electrical connection component 405 (e.g., a galvanic component), as described herein. Aspects of the diagram 400 may implement, or be implemented by, aspects of the diagrams 300-a and 300-b. In this regard, the electrical connection component 405 shown and described in FIG. 4 may be an example of the electrical connection component(s) 305 shown and described in FIGS. 3A and 3B.

In some cases, as described with reference to FIGS. 1 through 3B, the wearable ring device 104 may include an outer housing 410 and an inner housing 415 which may include electrically-conductive materials. The outer housing 410 and the inner housing 415 may be separated by a filler 430 (e.g., a filler material, such as an epoxy material, a plastic material), a side cover 420-a, and a side cover 420-b, which may be electrically-insulating materials. As such, the outer housing 410 and the inner housing 415 may be electrically insulated from each other, and the outer housing 410 and the inner housing 415 may develop separate electric potentials. If the electric potential difference is large enough, dielectric breakdown may occur, and current may flow between the outer housing 410 and the inner housing 415 through the filler 430 or other insulating components, which may damage components 425 (e.g., a PCB, capacitors, antenna components) of the wearable device 104.

In accordance with examples as described herein, the wearable device 104 may include the electrical connection component 405 (e.g., galvanic component) to reduce the risk of dielectric breakdown damaging the components 425. In some examples, as illustrated by the diagram 400, the electrical connection component 405 may be included within or adjacent to the side cover 420-a. For example, the electrical connection component 405 may be an electrically-conductive (e.g., metal) clip or sleeve that may be in contact with the outer housing 410 and the inner housing 415. As such, current may flow between the outer housing 410 and the inner housing 415 via the electrical connection component 405, which may reduce the effects of potential difference build-up between the outer housing 410 and the inner housing 415.

In some examples, while the diagram 400 illustrates the components 425 in the cross-section, the cross-section may instead include filler 430 and the components 425 may be located elsewhere within the wearable ring device 104. For example, it may be beneficial to place the electrical connection component 405 away from components that may be damaged by a surge of electric current flowing through the electrical connection component 405, or that may be subject to interference from the electrical connection component 405, as described herein. For example, in some cases, the electrical connection component 405 may be located along a portion of the circumference of the wearable ring device 104 that is non-overlapping with the portion of the circumference that includes the components 425 (e.g., electrical connection component 405 may be separated radially from the components 425).

FIG. 5 shows an example of a diagram 500 that supports techniques for electrostatic discharge protection for wearable devices in accordance with aspects of the present disclosure. The diagram 500 illustrates an example cross-section of a wearable ring device 104 that includes an electrical connection component 505 including a conductive body 501 and conductive flanges 520, which may be examples of electrical connection components or galvanic components as described herein. Aspects of the diagram 500 may implement, or be implemented by, aspects of the diagrams 300-a, 300-b, diagram 400, or any combination thereof. In this regard, the electrical connection component 505 shown and described in FIG. 5 may be an example of the electrical connection component(s) 305 and the electrical connection component 405 shown and described in FIGS. 3A and 3B and FIG. 4, respectively.

As described previously herein, the wearable ring device 104 may include an electrical connection component 505 that is configured to reduce a difference in an electrical potential between an inner housing 515 and an outer housing 510 by electrically coupling the inner housing 515 and the outer housing 510. For example, as shown in FIG. 5, the electrical connection component 505 may include a conductive body 501 and one or more conductive flanges 520.

In accordance with examples as described herein, the conductive body 501 may be in contact (e.g., electrically coupled) with the inner housing 515 of the wearable ring device 104, while the conductive flanges 520 may be in contact (e.g., electrically coupled) with the conductive body 501 and the outer housing 510 of the wearable ring device 104. As such, the inner housing 515 and the outer housing 510 may be electrically coupled with each other via the electrical connection component 505.

In some examples, the conductive flanges 520 may be flexible, such as to be in tension against the outer housing 510 to ensure electrical contact between the outer housing 510 and the conductive body 501. For example, the conductive flanges 520 may be flexible such that the conductive flanges 520 are pushed toward the inner housing 515 when the outer housing 510 is attached to the wearable ring device 104, thereby ensuring contact with the outer housing 510. In some cases, the conductive flanges 520 may include a center opening (e.g., a hole), such that the conductive body 501 may be inserted through the conductive flanges 520. In some examples, the conductive body 501 may be a pin or screw, and the conductive body 501 may include a head portion configured to prevent the conductive body 501 from fitting entirely through the center opening of the conductive flange 520. In some cases, the inner housing 515 may include an opening for the conductive body 501 and, in some examples, the opening may include threading for fixing the conductive body 501 (e.g., the screw). Additionally, or alternatively, the center opening of the conductive flanges 520 may include threading for fixing the conductive body 501.

In some examples, one or more flexible elements 525 may be included between portions of the conductive flanges 520. The flexible elements 525 may enhance the flexibility of the conductive flanges 520 or may facilitate a fitment of the conductive flanges 520 within the wearable ring device 104 (e.g., ensure that the conductive flanges 520 contact the outer housing 510). In some examples, the flexible elements 525 may extend to cover a sidewall of the conductive flanges 520. The flexible elements 525 may include rubber materials. Additionally, or alternatively, the flexible elements 525 may be or include other materials or parts, such as epoxy (e.g., epoxy blockers) or another polymer (e.g., Dryflex, Thermolast K, etc.).

While the electrical connection component 505 is shown and described in the context of a conductive body 501 and conductive flanges 520, it is contemplated herein that the electrical connection component 505 may be manufactured in additional or alternative designs. For example, in alternative cases, the electrical connection component 505 may include a conductive body 501 and a spring assembly coupled with the conductive body 501, where the spring assembly is configured to enable a conductive pin to move up and down relative to the inner housing 515 and to contact the outer housing 510. In this regard, the spring assembly may serve a same or similar purpose as the conductive flanges 520 shown and described in FIG. 5.

Accordingly, by including the conductive body 501 and the conductive flanges 520, the outer housing 510 and the inner housing 515 may be electrically coupled, thereby reducing the risk of damaging internal components of the wearable ring device 104 due to dielectric breakdown.

It should be noted that the methods described above describe possible implementations, and that the operations and the steps may be rearranged or otherwise modified and that other implementations are possible. Furthermore, aspects from two or more of the methods may be combined.

An apparatus device is described. The apparatus may include an inner housing component configured to at least partially contact a tissue of a user, the inner housing component comprising a first electrically-conductive material, an outer housing component that is at least partially exposed to a surrounding environment of the user, the outer housing component comprising a second electrically-conductive material, a PCB disposed at least partially between the inner housing component and the outer housing component, the PCB comprising one or more sensors configured to acquire physiological data from the user through one or more apertures within the inner housing component, and an electrical connection component configured to reduce a difference in an electrical potential between the inner housing component and the outer housing component by electrically coupling the inner housing component and the outer housing component.

Some examples of the apparatus may further include an antenna component configured to transmit and receive wireless signals, wherein a distance between the electrical connection component and the antenna component may be based at least in part on a wavelength associated with the wireless signals.

In some examples of the apparatus, the distance between the electrical connection component and the antenna component may be approximately equal to a quarter of the wavelength associated with the wireless signals.

In some examples of the apparatus, the antenna component comprises an antenna feed associated with a slot antenna.

In some examples of the apparatus, the PCB spans a first portion of a footprint of the wearable device and the electrical connection component may be positioned within a second portion of the footprint of the wearable device that may be separate from the first portion.

Some examples of the apparatus may further include a battery that spans a second radial portion around the circumference of the wearable ring device that may be separate from the first radial portion, wherein the electrical connection component may be located within a third portion of the circumference of the wearable ring device that may be between the first portion and the second portion.

Some examples of the apparatus may further include one or more side covers disposed between the inner housing component and the outer housing component, wherein the one or more side covers comprise one or more electrically insulating materials, and wherein the electrical connection component may be disposed within the one or more side covers.

In some examples of the apparatus, the one or more side covers comprise a first side cover disposed between the inner housing component and the outer housing component on a first lateral side of the wearable ring device and a second side cover disposed between the inner housing component and the outer housing component on a second lateral side of the wearable ring device opposite the first lateral side, wherein the first side cover and the second side cover comprise one or more electrically-insulating materials, and wherein the electrical connection component may be disposed within the first side cover, the second side cover, or both.

In some examples of the apparatus, the electrical connection component comprises a conductive body coupled to one of the inner housing component or the outer housing component and one or more conductive flanges that may be electrically coupled with and movable relative to the conductive body, the one or more conductive flanges configured to contact the other of the inner housing component or the outer housing component.

In some examples of the apparatus, the conductive body may be coupled to one of the inner housing component or the outer housing component via a screw, a rivet, a weld, solder, or any combination thereof.

In some examples of the apparatus, the electrical connection component comprises a conductive body coupled to one of the inner housing component or the outer housing component and a spring assembly that may be electrically coupled with and movable relative to the conductive body, the spring assembly configured to contact the other of the inner housing component or the outer housing component.

Some examples of the apparatus may further include an antenna component configured to transmit and receive wireless signals and one or more apertures within the inner housing component, the outer housing component, or both, the one or more apertures adjacent to the antenna component, wherein the one or more apertures enable the wireless signals to pass through the inner housing component, the outer housing component, or both.

In some examples of the apparatus, the first electrically conductive material comprises a first metallic material, the second electrically-conductive material comprises a second metallic material, and the second metallic material may be the same or different as the first metallic material.

In some examples of the apparatus, the wearable device comprises a wearable ring device. In some examples of the apparatus, the wearable device comprises a wrist-worn wearable device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

What is claimed is:

1. A wearable ring device, comprising:
an inner ring-shaped housing configured to at least partially contact a tissue of a user, the inner ring-shaped housing comprising a first electrically-conductive material;
an outer ring-shaped housing that is at least partially exposed to a surrounding environment of the user, the outer ring-shaped housing comprising a second electrically-conductive material;
a printed circuit board (PCB) disposed at least partially between the inner ring-shaped housing and the outer ring-shaped housing, where in the PCB spans a first radial portion of the wearable ring device, the PCB comprising one or more sensors configured to acquire physiological data from the user through one or more apertures within the inner ring-shaped housing;
a curved battery that spans a second radial portion of the wearable ring device that is separate from the first radial portion, the curved battery positioned between the inner ring-shaped housing and the outer ring-shaped housing; and
an electrical connection component configured to reduce a difference in an electrical potential between the inner ring-shaped housing and the outer ring-shaped housing by electrically coupling the inner ring-shaped housing and the outer ring-shaped housing, wherein the electrical connection component is located within a third radial portion of the wearable ring device that is between the first radial portion and the second radial portion.

2. The wearable ring device of claim 1, further comprising:
an antenna component configured to transmit and receive wireless signals, wherein a distance between the electrical connection component and the antenna component is based at least in part on a wavelength associated with the wireless signals.

3. The wearable ring device of claim 2, wherein the distance between the electrical connection component and the antenna component is approximately equal to a quarter of the wavelength associated with the wireless signals.

4. The wearable ring device of claim 2, wherein the antenna component comprises an antenna feed associated with a slot antenna.

5. The wearable ring device of claim 1, wherein the electrical connection component comprises:
a conductive body coupled to one of the inner ring-shaped housing or the outer ring-shaped housing; and
one or more conductive flanges that are electrically coupled with and movable relative to the conductive body, the one or more conductive flanges configured to contact the other of the inner ring-shaped housing or the outer ring-shaped housing.

6. A wearable device, comprising:
an inner housing configured to at least partially contact a tissue of a user, the inner housing comprising a first electrically-conductive material;
an outer housing that is at least partially exposed to a surrounding environment of the user, the outer housing comprising a second electrically-conductive material;
a printed circuit board (PCB) disposed at least partially between the inner housing and the outer housing, the PCB comprising one or more sensors configured to acquire physiological data from the user through one or more apertures within the inner housing; and
an electrical connection component configured to reduce a difference in an electrical potential between the inner housing and the outer housing by electrically coupling the inner housing and the outer housing.

7. The wearable device of claim 6, further comprising:
an antenna component configured to transmit and receive wireless signals, wherein a distance between the electrical connection component and the antenna component is based at least in part on a wavelength associated with the wireless signals.

8. The wearable device of claim 7, wherein the distance between the electrical connection component and the antenna component is approximately equal to a quarter of the wavelength associated with the wireless signals.

9. The wearable device of claim 7, wherein the antenna component comprises an antenna feed associated with a slot antenna.

10. The wearable device of claim 6, wherein the PCB spans a first portion of a footprint of the wearable device, and wherein the electrical connection component is positioned within a second portion of the footprint of the wearable device that is separate from the first portion.

11. The wearable device of claim 6, wherein the wearable device comprises a wearable ring device, wherein the PCB spans a first radial portion around a circumference of the wearable ring device, the wearable device further comprising:
a battery that spans a second radial portion around the circumference of the wearable ring device that is separate from the first radial portion, wherein the electrical connection component is located within a third radial portion of the circumference of the wearable ring device that is between the first radial portion and the second radial portion.

12. The wearable device of claim 6, further comprising:
one or more side covers disposed between the inner housing and the outer housing, wherein the one or more side covers comprise one or more electrically-insulating materials, and wherein the electrical connection component is disposed within the one or more side covers.

13. The wearable device of claim 12, wherein the wearable device comprises a wearable ring device, and wherein the one or more side covers comprise:
a first side cover disposed between the inner housing and the outer housing on a first lateral side of the wearable ring device; and
a second side cover disposed between the inner housing and the outer housing on a second lateral side of the wearable ring device opposite the first lateral side, wherein the first side cover and the second side cover comprise one or more electrically-insulating materials, and wherein the electrical connection component is disposed within the first side cover, the second side cover, or both.

14. The wearable device of claim 6, wherein the electrical connection component comprises:
a conductive body coupled to one of the inner housing or the outer housing; and
one or more conductive flanges that are electrically coupled with and movable relative to the conductive body, the one or more conductive flanges configured to contact the other of the inner housing or the outer housing.

15. The wearable device of claim 14, wherein the conductive body is coupled to one of the inner housing or the outer housing via a screw, a rivet, a weld, solder, or any combination thereof.

16. The wearable device of claim 6, wherein the electrical connection component comprises:
a conductive body coupled to one of the inner housing or the outer housing; and
a spring assembly that is electrically coupled with and movable relative to the conductive body, the spring assembly configured to contact the other of the inner housing or the outer housing.

17. The wearable device of claim 6, further comprising:
an antenna component configured to transmit and receive wireless signals; and
one or more apertures within the inner housing, the outer housing, or both, the one or more apertures adjacent to the antenna component, wherein the one or more apertures enable the wireless signals to pass through the inner housing, the outer housing, or both.

18. The wearable device of claim 6, wherein the first electrically-conductive material comprises a first metallic material, the second electrically-conductive material comprises a second metallic material, and the second metallic material is the same or different as the first metallic material.

19. The wearable device of claim 6, wherein the wearable device comprises a wearable ring device.

20. The wearable device of claim 6, wherein the wearable device comprises a wrist-worn wearable device.
